(19) United States
(12) Patent Application Publication
(10) Pub. No.: US 2021/0201492 A1
(43) Pub. Date: Jul. 1, 2021

(54) IMAGE-BASED SKIN DIAGNOSTICS

(71) Applicant: L'OREAL, Paris (FR)

(72) Inventor: Kyle Yeates, Redmond, WA (US)

(73) Assignee: L'OREAL, Paris (FR)

(21) Appl. No.: 17/138,352

(22) Filed: Dec. 30, 2020

Related U.S. Application Data
(60) Provisional application No. 62/955,159, filed on Dec. 30, 2019.

(51) Int. Cl. G06T 7/00; G06T 7/80; H04N 17/00; G06T 7/90; G16H 30/20; G16H 50/20; G16H 20/10; A45D 44/00

(52) U.S. Cl. CPC G06T 7/0016; G06T 7/80; H04N 17/002; G06T 7/90; G16H 30/20; A45D 2044/007; G16H 20/10; A45D 44/005; G06T 2207/30088; G06T 2207/10024; G16H 50/20

(57) ABSTRACT

Examples of the present disclosure relate to systems and methods for generating more accurate image(s) of a user via a camera of a consumer product (e.g., mobile phone, tablet, laptop, etc.) for subsequent use in, for example, computer implemented applications, such as skin diagnosis, facial recognition, cosmetic simulation, selection and/or recommendation, etc. Examples of the systems and methods improve image accuracy and quality by addressing issues relating to unpredictable and inconsistent lighting conditions, among others. In an example, the system includes a mobile computing device and an object with known lighting and/or color attributes (e.g., a reference). Such an object acts as a calibration device for images to be captured by the mobile computing device.

### CROSS-REFERENCE TO RELATED APPLICATIONS

This application claims the benefit of U.S. Provisional Application No. 62/955,159, filed Dec. 30, 2019, the disclosure of which is incorporated herein in its entirety.

### TECHNICAL FIELD

Embodiments of the present disclosure relate to image processing. In some embodiments, image processing techniques are employed for skin condition diagnostics and/or treatment. In order to provide improved image processing, calibration techniques can be employed.

### SUMMARY OF THE DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with an aspect of the disclosure, a computer implemented method for accurate skin diagnosis is provided. In an embodiment the method comprises calibrating, by a computing device, one or more images of an area of interest associated with a subject; and determining a skin condition based on the one or more calibrated images.

In any embodiment, the one or more images includes a plurality of images taken sequentially over a period of time, and wherein said determining a skin condition is based on the plurality of images

In any embodiment, the method may further comprises generating a treatment protocol and/or product recommendation for an area of interest of the subject based on the determined skin condition.

In any embodiment, calibrating, by a computing device, one or more images of an area of interest associated with a subject includes obtaining calibration data from a calibration device; calibrating a camera based on the calibration data; and capturing the one or more images of a user with the calibrated camera.

In any embodiment, the method may further comprise generating, via the calibration device, light meter data or color temperature data of the subject; receiving the calibration data from the calibration device; and adjusting one or more camera settings for calibrating the camera prior to image capture.

In any embodiment, calibrating, by a computing device, one or more images of an area of interest associated with a subject includes capturing the one or more images via a camera associated with the computing device; obtaining calibration data from a calibration device associated with the one or more images captured by the camera; and calibrating the one or more images captured by the camera based on the calibration data. In any embodiment, the method may further comprises generating, via the calibration device, light meter data or color temperature data of the subject; receiving the light meter data and/or color meter data from the calibration device, and using said light meter data and/or color meter data obtained from the calibration device to calibrate the captured images.

In any embodiment, the calibration device includes one selected from the group consisting of a color card, a color chip and a calibration reference, the method further comprising capturing at least one image of the subject in the presence of the calibration device.

In any embodiment, the calibration device is a cosmetics apparatus.

In accordance with another embodiment, a method is provided, comprising obtaining calibration data from a calibration device; generating, by a computing device, calibrated images by one of: calibrating a camera of a mobile computing device based on the calibration data and capturing one or more images of a user with the calibrated camera; or calibrating one or more images captured with the camera based on the calibration data.

In any embodiment, the method may further comprise determining a skin condition based on the one or more calibrated images.

In any embodiment, the method may further comprise recommending one or more of: a skin treatment protocol; and a product configured to treat the skin condition.

In accordance with another embodiment, a computer system is provided. The system includes a user interface engine including circuitry configured to cause an image capture device to capture images of the user; a calibration engine including circuitry configured to calibrate the image capture device prior to image capture for generating calibrated images or to calibrate the images captured by the image capture device for generating calibrated images, said calibration engine obtaining calibration data from a calibration device; and a skin condition engine configured to determine a skin condition of the user based on the generated calibrated images image.

In any embodiment, the system may further comprise a recommendation engine including circuitry configured to recommend a treatment protocol or a product based at least on the determined skin condition.

In any embodiment, calibration device includes one or more sensors configured to generate data indicative of calibration data, and wherein the calibration engine is configured to receive the calibration data and adjust one or more suitable camera settings for calibrating the camera prior to image capture.

In any embodiment, the calibration device includes an attribute suitable for use by the calibration engine to generate the calibrated images.

In any embodiment, the attribute is a color or an indicia indicative of a color, the calibration engine configured to obtain calibration data based on the indicia.

In any embodiment, the calibration engine includes circuitry configured to obtain the calibration data from the image captured by the image capture device, the image captured including an image of the calibration device.

In any embodiment, the calibration device is a cosmetics apparatus or packaging associated therewith.

In any embodiment, the calibration engine is configured to: automatically detect a color reference associated with the captured image; and use the color reference in order to correct the colors of the captured image to generate calibrated images.

### DESCRIPTION OF THE DRAWINGS

The foregoing aspects and many of the attendant advantages of disclosed subject matter will become more readily appreciated as the same become better understood by reference to the following detailed description, when taken in conjunction with the accompanying drawings, wherein:

FIG. 1 is a schematic diagram that illustrates a non-limiting example of a system for calibrating images of a user according to an aspect of the present disclosure, the calibrated images being suitable for use in applications such as diagnosing skin conditions, facial recognition, cosmetic recommendations, etc.;

FIG. 2 is a block diagram that illustrates a non-limiting example of a mobile computing device according to various aspects of the present disclosure;

FIG. 3 is a block diagram that illustrates a non-limiting example of a server computing device according to an aspect of the present disclosure;

FIG. 4 is a block diagram that illustrates a non-limiting example of a computing device appropriate for use as a computing device with embodiments of the present disclosure.

FIG. 5 is a flowchart that illustrates a non-limiting example of a method for generating calibrated images according to an aspect of the present disclosure.

### DETAILED DESCRIPTION

Examples of methodologies and technologies for improved image capture for use in various applications, such as skin diagnosis, product selection, facial recognition, etc., are described herein. Thus, in the following description, numerous specific details are set forth to provide a thorough understanding of the examples. One skilled in the relevant art will recognize; however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one example” or “one embodiment” means that a particular feature, structure, or characteristic described in connection with the example is included in at least one example of the present invention. Thus, the appearances of the phrases “in one example” or “in one embodiment” in various places throughout this specification are not necessarily all referring to the same example. Furthermore, the particular features, structures, or characteristics may be combined in any suitable manner in one or more examples.

Examples of the present disclosure relate to systems and methods for generating more accurate image(s) of a user via a camera of a consumer product (e.g., mobile phone, tablet, laptop, etc.) for subsequent use in, for example, computer implemented applications, such as skin diagnosis, facial recognition, cosmetic simulation, selection and/or recommendation, etc. Examples of the systems and methods improve image accuracy and quality by addressing issues relating to unpredictable and inconsistent lighting conditions, among others. In an example, the system includes a mobile computing device and an object with known lighting and/or color attributes (e.g., a reference). Such an object acts as a calibration device for images to be captured by the mobile computing device.

In some examples, the calibration device can provide light or color meter data, color card data, color reference data or other calibration data to the mobile computing device. By accessing or receiving calibration data from the calibration device, the mobile computing device can generate calibrated images to compensate for non-uniform lighting conditions, for example. In some embodiments, the calibration data can be used prior to image capture for camera setting(s) adjustment. In other embodiments, the calibration data can be alternatively used after image capture for calibrating the images when the captured images are processed for storage.

In some examples, the methodologies and technologies are carried out by a computing system that includes, for example, a handheld smart device (e.g., a smart phone, tablet, laptop, game console, etc.) with a camera and memory. An optional cloud data store can be accessed by the system for storage of images of the user with appropriate metadata (e.g., date, camera settings, user ID, etc.). The computing system also includes one or more image processing algorithms or engines that are either local to the handheld smart device or remote to the handheld smart device (e.g., server/cloud system) for analyzing the captured images.

In some examples, the methodologies and technologies of the disclosure are provided to a user as a computer application (i.e., an “App”) through a mobile computing device, such as a smart phone, a tablet, a wearable computing device, or other computing devices that are mobile and are configured to provide an App to a user. In other examples, the methodologies and technologies of the disclosure may be provided to a user on a computer device by way of a network, through the Internet, or directly through hardware configured to provide the methodologies and technologies to a user.

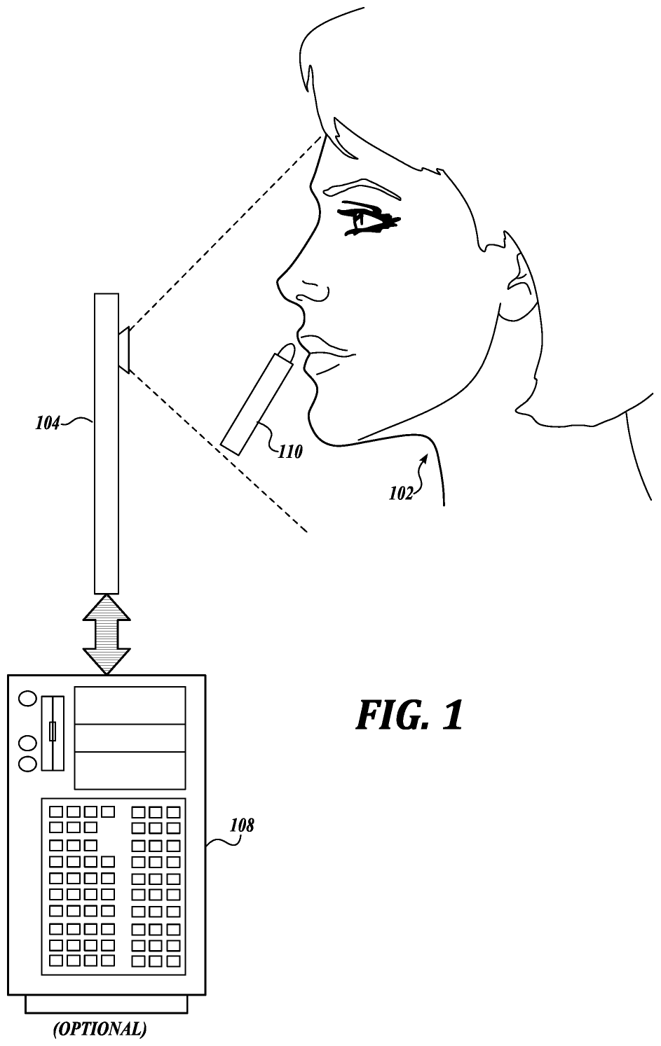

FIG. 1 is a schematic diagram that illustrates a non-limiting example of a system 100 for generating calibrated images of a user according to various aspects of the present disclosure. In some embodiments, the system 100 may use the calibrated images for diagnosing skin condition of a user, for example. In other embodiments, the system can use the calibrated images for facial recognition applications.

In yet other embodiments, the calibrated images may be utilized for generating a recommendation for cosmetic products. For example, such cosmetic products may be for skin, anti-aging, face, nails, and hair, or any other beauty or health product. As a further example, products may include creams, cosmetics, nail polish, shampoo, conditioner, other hair products, vitamins, any health-related products of any nature, or any other product that offer results visible in a person's appearance, such as a person's skin, hair, nails or other aspects of a person's appearance. Examples of treatments may include diet treatments, physical fitness treatments, acupuncture treatments, acne treatments, appearance modification treatments, or any other treatment that offers results visible in a person's appearance.

For more information on suitable uses for the calibrated data, all of which are within the scope of and are embodiments of the disclosure, please see U.S. Pat. No. 9,760,925, the disclosure of which is incorporated by reference in its entirety.

In the system 100, a user 102 interacts with a mobile computing device 104 and a calibration device 106. In one example, the mobile computing device 104 is used to capture one or more images of the user 102 in the presence of the calibration device 106. The calibration device 106 is associated with or generates calibration data, such as light meter data, color meter data, color card (e.g., color reference) data, etc. The calibration data is used by the system 100 to generate calibrated images via the mobile computing device 104, for example. In an example, the calibration device 106 can be used to calibrate the mobile computing device 104 (e.g., a camera of the mobile computing device) prior to image capture in order to generate calibrated images. In other embodiments, the calibration data can be used by the mobile computing device 104 after image capture for generating calibrated images. Because of the calibration data provided by the calibration device, the images can be either captured or processed in a way to obtain, for example, true colors of the user, regardless of the lighting conditions, etc., in which the image was taken.

In the embodiment shown, the calibration device 106 is a cosmetic, such as lipstick. In this embodiment, the cosmetic packaging includes one or more colors that can be used as a color calibration reference. In some embodiments, the color(s) is chosen from a list of colors from the Macbeth chart. Generally, the Macbeth chart is comprised of a number of colors with known color values. Other color reference systems can be also used. In some embodiments, the color(s) can be on the exterior of the cosmetic packaging or on a part thereof (e.g., cap, lid, etc.) that can be visible to the mobile computing device 104.

In some embodiments, the calibration data of the calibration device 106 can be associated with other material obtained at the point of sale, for example, the box or other container/packaging, the product literature, etc. In some embodiments, the associated material includes a color card, or parts thereof, for example. The color card can include colors, for example, of the Macbeth chart. In other embodiments, the associated material includes one or more colors and/or associated indicia. The associated indicia (e.g., QR code, bar code, symbol, etc.) can be used by the system to obtain, for example, the color value(s) of the one or more colors included in the associated material or of the cosmetic packaging for calibration purposes. In one example, the associated indicia can be linked to color value(s) in a calibration data store.

In some embodiments, the calibration device 106 includes one or more sensors configured to generate color meter data, light meter data, etc. For example, the calibration device 106 in one embodiment includes one or more photosensors (e.g., photodiodes) configured to sense light conditions and generate light calibration data. Additionally or alternatively, the calibration device 106 in other embodiments includes one or more photosensors (e.g., filtered photodiodes) configured to sense color temperature and generate color calibration data. In other embodiments, the mobile device may include such sensors, and may be used to capture such calibration affecting data.

Of course, the calibration device 106 can take many forms or functions. For example, the calibration device 106 can be a cosmetic, such as a lipstick, eyeshadow, foundation, etc., a hair brush, a toothbrush, etc., or an appliance, such as a Clarisonic branded skin care appliance. In other embodiments, the only function of the calibration device 106 is to provide calibration data.

In some embodiments, the calibration device 106 is configured to transmit the calibration data to the mobile computing device 104. In some embodiments, the calibration device 106 can be coupled (e.g., wired or wirelessly) in data communication with the mobile computing device 104 according to any known or future developed protocol, such as universal serial bus, Bluetooth, WiFi, Infrared, ZigBee, etc. In an embodiment, the calibration device 106 includes a transmitter for transmitting the calibration data.

In some embodiments, once the calibration device 106 is turned on and in range of the mobile computing device 104, it automatically pairs and sends the calibration data to the mobile computing device 104. In other embodiments, the mobile computing device 104 pulls the calibration data from the calibration device 106 via a request or otherwise. In yet other embodiments, the mobile computing device 104 obtains the calibration data from a local data store or a remote data store, such as the calibration data store, based on the associated indicia of the calibration device 106.

As will be described in more detail below, the mobile computing device 104 in some embodiments can carry out a device calibration routine to adjust camera settings, such as white balance, brightness, contrast, exposure, aperture, flash, etc., based on the provided calibration data prior to image capture. As will be also described in more detail below, the calibration data can be also used after image capture in some embodiments. For example, an image captured along with calibration data can be adjusted via imaging processing. In one embodiment in which color data is obtained via a color reference, the image can be compared to a reference image that also contains the color reference with the same color value(s). From the comparison(s), various attributes of the image(s) can be adjusted to calibrate the image. In other embodiments, the calibration device includes associated indicia that can be used to retrieve color values of the calibration device. From the retrieved color value(s), various attributes of the image(s) can be adjusted to calibrate the captured image. In yet other embodiments, the calibration device can transmit light and/or color meter data to the mobile computing device. With the light and/or color meter data, calibrated images are generated by the mobile computing device from the captured images.

As a result, the images captured and/or processed by the mobile computing device 104 would look the same whether the user has taken the photo in a dark room, a bright room, or a room with non-uniform and highly angled lighting. Thus, calibrated images are generated by the mobile computing device. This standardization process can lead to a reduction or elimination in the variability in the quality of images used for applications ranging from diagnosing skin conditions and/or cosmetic recommendations to facial recognition, for example.

As will be described in more detail below, some of the functionality of the mobile computing device 104 can be additionally or alternatively carried out at an optional server computing device 108. For example, the mobile computing device 104 in some embodiments transmits the captured images to the server computing device 108 via a network 110 for image processing (e.g., calibration, skin condition diagnosis, product recommendation, facial recognition, etc.) and/or storage. In some embodiments, the network 110 may include any suitable wireless communication technology (including but not limited to Wi-Fi, WiMAX, Bluetooth, 2G, 2G, 4G, 5G, and LTE), wired communication technology (including but not limited to Ethernet, USB, and FireWire), or combinations thereof.

For example, with the captured images received from the mobile computing device 104, the server computing device 108 may process the captured images for calibration purposes and/or store the calibrated images for subsequent retrieval. In other embodiments, calibrated images are transmitted to the server computing device 108 for storage and/or further processing, such as skin condition diagnosis, etc. In some embodiments, the server computing device 108 can serve calibration data to the mobile computing device 104 for local processing.

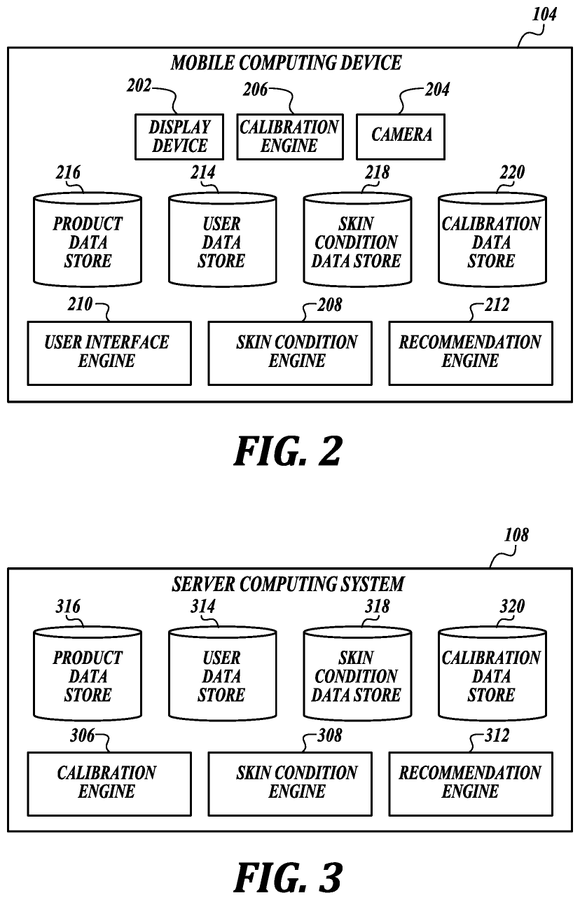

FIG. 2 is a block diagram that illustrates a non-limiting example of a mobile computing device 104 according to an aspect of the present disclosure. In some embodiments, the mobile computing device 104 may be a smartphone. In some embodiments, the mobile computing device 104 may be any other type of computing device having the illustrated components, including but not limited to a tablet computing device or a laptop computing device. In some embodiments, the mobile computing device 104 may not be mobile, but may instead by a stationary computing device such as a desktop computing device or computer kiosk. In some embodiments, the illustrated components of the mobile computing device 104 may be within a single housing. In some embodiments, the illustrated components of the mobile computing device 104 may be in separate housings that are communicatively coupled through wired or wireless connections (such as a laptop computing device with an external camera connected via a USB cable). The mobile computing device 104 also includes other components that are not illustrated, including but not limited to one or more processors, a non-transitory computer-readable medium, a power source, and one or more communication interfaces. As shown, the mobile computing device 104 includes a display device 202, a camera 204, a calibration engine 206, a skin condition engine 208, a user interface engine 210, a recommendation engine 212 (optional), and one or more data stores, such as a user data store 214, a product data store 216 and/or skin condition data store 218, and a calibration data store 220. Each of these components will be described in turn.

In some embodiments, the display device 202 is an LED display, an OLED display, or another type of display for presenting a user interface. In some embodiments, the display device 202 may be combined with or include a touch-sensitive layer, such that a user 102 may interact with a user interface presented on the display device 202 by touching the display. In some embodiments, a separate user interface device, including but not limited to a mouse, a keyboard, or a stylus, may be used to interact with a user interface presented on the display device 202.

In some embodiments, the user interface engine 210 is configured to present a user interface on the display device 202. In some embodiments, the user interface engine 210 may be configured to use the camera 204 to capture images of the user 102. For example, the user 102 may take a “selfie” with the mobile computing device 104 via camera 204. Of course, a separate image capture engine may also be employed to carry out at least some of the functionality of the user interface 210. The user interface presented on the display device 202 can aid the user in capturing images, storing the captured images, accessing the previously stored images, interacting with the other engines, etc.

In some embodiments, the camera 204 is any suitable type of digital camera that is used by the mobile computing device 104. In some embodiments, the mobile computing device 104 may include more than one camera 212, such as a front-facing camera and a rear-facing camera. In some embodiments, the camera 204 includes adjustable settings, such as white balance, brightness, contrast, exposure, aperture, and/or flash, etc. Generally herein, any reference to images being utilized by the present disclosure, should be understood to reference both video, images (one or more images), or video and images (one or more images), as the present disclosure is operable to utilize video, images (one or more images), or video and images (one or more images) in its methods and systems described herein.

In some embodiments, the calibration engine 206 is configured to calibrate the camera 204 of the mobile computing device 104 based on calibration data obtained from at least one of the calibration device 106 or the calibration data store 220. In some embodiments, the calibration engine 206 is configured to adjust the settings of the camera 204 prior to image capture. In other embodiments, instead of calibrating the camera 204 prior to image capture, the calibration engine 206 is configured to calibrate the images after image capture. For example, calibration data from the calibration device 206 can be used when processing the captured images prior to or during storage.

In some embodiments, the calibration engine 206 detects a color reference (such as a color card) within the captured image and uses the color reference in order to correct the colors of the captured image for calibration purposes. For example, the calibration engine 206 in some embodiments compares the image captured by the camera 204 to a reference image stored in the calibration data store 220. The reference image contains some of, all of, etc., the color calibration data of the captured image. For example, the calibration device 106 (e.g., cosmetic packaging, product literature, appliance handle, etc.) in the captured image may include a color card, a color chip, or other color reference, etc., to be compared to the reference image stored in calibration data store 220. In other embodiments, the color of the calibration device 106 has a known color value. In yet other embodiments, the calibration device 106 includes one or more colors with a known color value that can be retrieved from the calibration data store 220 via indicia visibly associated with the calibration device 106. In some embodiments, the color reference detected by the calibration engine 206 within the captured image is indicia that can be used to retrieve the color value(s) from the calibration data store 220 in order to correct the colors of the captured image for calibration purposes. In yet other embodiments, the data representing the known colors can be used to adjust one or more settings (e.g., white balance, brightness, color values, etc.) of the camera for subsequent image capture.

After calibration, the calibrated images are saved in a data store, such as user data store 214, and can be subsequently used for product selection (e.g., hair color, lipstick color, eye shadow color, etc.), diagnosis, such as skin condition, or for other purposes such as facial recognition applications.

The mobile computing device 104 may be provided with other engines for increased functionality. For example, in the embodiment shown, the mobile computing device 104 includes a skin condition engine 208. The skin condition engine 208 is configured to analyze the calibrated images to determine one or more skin conditions (e.g., acne, eczema, psoriasis, etc.) of the user 102. The skin condition engine 208 may retrieve data from the skin condition data store 218 during the analysis. In some of these embodiments, a recommendation engine 212 may also be provided, which recommends a treatment protocol, products for treatment, etc., based on the results of the analysis carried out by the skin condition engine 208. In doing so, the recommendation engine 212 can access data from the product data store 216.

In other embodiments, a facial recognition engine (not shown) is provided, which is configured to identify the identity of or other attribute of the user. In yet other embodiments, a cosmetic recommendation engine (not shown) is provided, which can simulate product color, such as hair color, lipstick, etc., on the user for aid in product selection, product recommendation, etc. In some embodiments, the cosmetic recommendation engine is part of the recommendation engine 212 and can access data from the product data store 216. Any recommendation generated by the recommendation engine 212 can be presented to the user in any fashion via the user interface engine 210 on display 202.

Further details about the actions performed by each of these components are provided below.

“Engine” refers to refers to logic embodied in hardware or software instructions, which can be written in a programming language, such as C, C++, COBOL, JAVA™ PHP, Perl, HTML, CSS, JavaScript, VBScript, ASP, Microsoft .NET™, Go, and/or the like. An engine may be compiled into executable programs or written in interpreted programming languages. Software engines may be callable from other engines or from themselves. Generally, the engines described herein refer to logical modules that can be merged with other engines, or can be divided into sub-engines. The engines can be stored in any type of computer-readable medium or computer storage device and be stored on and executed by one or more general purpose computers, thus creating a special purpose computer configured to provide the engine or the functionality thereof.

“Data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

FIG. 3 is a block diagram that illustrates various components of a non-limiting example of an optional server computing system 108 according to an aspect of the present disclosure. In some embodiments, the server computing system 108 includes one or more computing devices that each include one or more processors, non-transitory computer-readable media, and network communication interfaces that are collectively configured to provide the components illustrated below. In some embodiments, the one or more computing devices that make up the server computing system 108 may be rack-mount computing devices, desktop computing devices, or computing devices of a cloud computing service.

In some embodiments, image processing and/or storage of the captured images can be additionally or alternatively carried out at an optional server computing device 108. In that regard, the server computing device 108 can receive captured and/or processed images from the mobile computing device 104 over the network 110 for processing and/or storage. As shown, the server computing device 108 optionally includes a calibration engine 306, a skin condition engine 308, a recommendation engine 312, and one or more data stores, such as a user data store 314, a product data store 316, a skin condition data store 318, and/or a calibration data store 320. It will be appreciated that the calibration engine 306, the skin condition engine 308, the recommendation engine 312, and the one or more data stores, such as the user data store 314, the product data store 316, the skin condition data store 318, and/or the calibration data store 320 are substantially identical in structure and functionality as the calibration engine 206, the skin condition engine 208, the recommendation engine 212, and one or more data stores, such as the user data store 214, the product data store 216, the skin condition data store 218, and/or the calibration data store 220 of the mobile computing device 104 illustrated in FIG. 2.

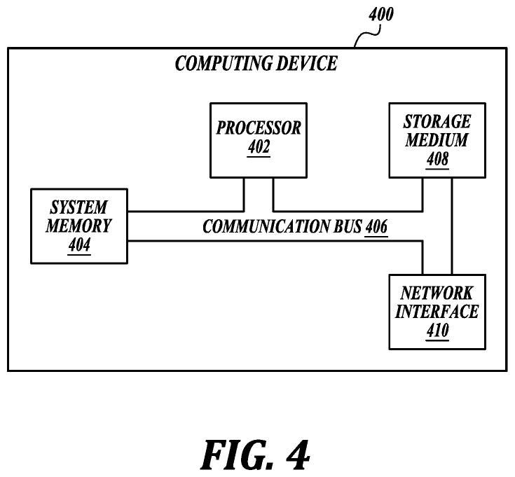

FIG. 4 is a block diagram that illustrates aspects of an exemplary computing device 400 appropriate for use as a computing device of the present disclosure. While multiple different types of computing devices were discussed above, the representative computing device 400 describes various elements that are common to many different types of computing devices. While FIG. 4 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Moreover, those of ordinary skill in the art and others will recognize that the computing device 400 may be any one of any number of currently available or yet to be developed devices.

In its most basic configuration, the computing device 400 includes at least one processor 402 and a system memory 404 connected by a communication bus 406.

Depending on the exact configuration and type of device, the system memory 404 may be volatile or nonvolatile memory, such as read only memory (“ROM”), random access memory (“RAM”), EEPROM, flash memory, or similar memory technology. Those of ordinary skill in the art and others will recognize that system memory 404 typically stores data and/or program modules that are immediately accessible to and/or currently being operated on by the processor 402. In this regard, the processor 402 may serve as a computational center of the computing device 400 by supporting the execution of instructions.

As further illustrated in FIG. 4, the computing device 400 may include a network interface 410 comprising one or more components for communicating with other devices over a network. Embodiments of the present disclosure may access basic services that utilize the network interface 410 to perform communications using common network protocols. The network interface 410 may also include a wireless network interface configured to communicate via one or more wireless communication protocols, such as WiFi, 2G, 2G, LTE, WiMAX, Bluetooth, Bluetooth low energy, and/or the like. As will be appreciated by one of ordinary skill in the art, the network interface 410 illustrated in FIG. 4 may represent one or more wireless interfaces or physical communication interfaces described and illustrated above with respect to particular components of the computing device 400.

In the exemplary embodiment depicted in FIG. 4, the computing device 400 also includes a storage medium 408. However, services may be accessed using a computing device that does not include means for persisting data to a local storage medium. Therefore, the storage medium 408 depicted in FIG. 4 is represented with a dashed line to indicate that the storage medium 408 is optional. In any event, the storage medium 408 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer readable instructions, data structures, program modules, or other data. In this regard, the system memory 404 and storage medium 408 depicted in FIG. 4 are merely examples of computer-readable media.

Suitable implementations of computing devices that include a processor 402, system memory 404, communication bus 406, storage medium 408, and network interface 410 are known and commercially available. For ease of illustration and because it is not important for an understanding of the claimed subject matter, FIG. 4 does not show some of the typical components of many computing devices. In this regard, the computing device 400 may include input devices, such as a keyboard, keypad, mouse, microphone, touch input device, touch screen, tablet, and/or the like. Such input devices may be coupled to the computing device 400 by wired or wireless connections including RF, infrared, serial, parallel, Bluetooth, Bluetooth low energy, USB, or other suitable connections protocols using wireless or physical connections. Similarly, the computing device 400 may also include output devices such as a display, speakers, printer, etc. Since these devices are well known in the art, they are not illustrated or described further herein.

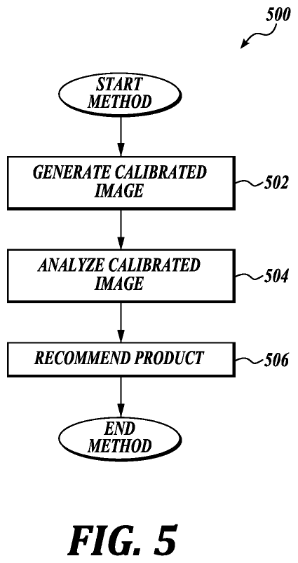

FIG. 5 is a flowchart that illustrates a non-limiting example embodiment of a method 500 for calibrating images of a user according to an aspect of the present disclosure. It will be appreciated that the following method steps can be carried out in any order or at the same time, unless an order is set forth in an express manner or understood in view of the context of the various operation(s). Additional process steps can also be carried out. Of course, some of the method steps can be combined or omitted in example embodiments.

From a start block, the method 500 proceeds to block 502, where calibrated images are generated by the mobile computing device 104 and/or the server computing system 108 with the aid of calibration data from the calibration device 106. For example, the user 102 can operate the calibration device 106 in some embodiments to generate data indictive of, for example, ambient lighting conditions. The calibration device 106 may additionally or alternatively generate color temperature data of the user 102. For example, in one embodiment in which the calibration device 106 includes one or more photosensors, the user 102 can scan an area of interest (e.g., face) with a sweeping movement. This can occur, for example, during a face cleansing or make-up application/removal routine just prior to, contemporaneously with, or just after image capture by the mobile computing device 104. During the scan, the calibration device 106 records light meter data generated by the photosensor(s). If equipped, the calibration device 106 alternatively or additionally records color meter data of the user via an appropriate sensor. The light meter data and/or color meter data can then be transferred (wired or wirelessly) to the mobile computing device 104 and/or server computing system 108.

With the generated light meter data and/or color meter data, the calibration engine can calibrate either the camera 204 of the mobile computing device 104 or the images captured by the camera. For example, the mobile computing device 104 can receive the calibration data (e.g., light meter data, color meter data, etc.) from the calibration device 106 via any wired or wireless protocol and adjust the appropriate camera settings to calibrate the camera 204 prior to image capture. With the calibrated camera, the mobile computing device can generate calibrated image(s) of an area of interest of the user 102. Alternatively, the calibration engine can use the light meter data and/or color meter data obtained from the calibration device 106 to calibrate the images captured by the camera 204.

In some embodiments, the images captured are of an area of interest to the user 102. For example, the area of interest can be one of face, the neck, the arm, etc., for tracking skin conditions, such as moles, sun spots, acne, eczema, etc.

In another embodiment, an attribute of the calibration device 106 can be used by the calibration engine to generate calibrated images. In this embodiment, the mobile computing device 104 captures at least one image of the user 102 in the presence of the calibration device 106. In some embodiments, the at least one image to be captured is of an area of interest to the user 102. For example, the area of interest can be one of face, the neck, the arm, etc., for tracking skin conditions, such as lesions, moles, sun spots, acne, eczema, etc.

For example, the user 102 can capture an image of themselves (a “selfie”) holding the calibration device 106. In this embodiment, the calibration device 106 may include a color card, a color chip or other feature that can provide a reference for calibration purposes. From the captured image, the calibration engine 206 can extract calibration data and can then generate a calibrated image. In some embodiments, the calibrated image is generated by adjusting the appropriate camera settings to calibrate the camera 204. With the calibrated camera settings, the mobile computing device generates calibrated images. For example, the user interface engine captures an image to be used for calibration purposes. Once calibrated, the camera can be used to capture calibrated images for skin condition applications, facial recognition applications, etc. In some other embodiments, a calibrated image is generated via image processing techniques by adjusting one or more image attributes (e.g., white balance, brightness, color values, etc.) of the image after image capture.

In some embodiments, the calibration engine automatically detects a color reference (such as a color card) within the captured image and uses the color reference in order to correct the colors of the captured image for calibration purposes. For example, the calibration engine in some embodiments compares the image captured by the camera 204 to a reference image stored in the calibration data store 220, 320. The reference image contains some of, all of, etc., the color calibration data of the captured image. For example, the calibration device 106 (e.g., cosmetic packaging, product literature, appliance handle, etc.) in the captured image may include a color card, a color chip, or other color reference, etc., to be compared to the reference image stored in calibration data store.

In other embodiments, the color of the calibration device 106 has a known color value. In yet other embodiments, the calibration device 106 includes one or more colors with a known color value that can be retrieved from the calibration data store via indicia visibly associated with the calibration device 106. In some embodiments, the color reference detected by the calibration engine 206 within the captured image is indicia that can be used to retrieve the color value(s) from the calibration data store 220 in order to correct the colors of the captured image for calibration purposes.

The calibrated images generated by the calibration engine are then stored in the user data store 214 of the mobile computing device 104 for subsequent retrieval. During storage of the captured images of the user, additional image processing (e.g., filtering, transforming, compressing, etc.) can be undertaken, if desired. Additionally or alternatively, the captured images can be transferred to the server computing device 108 over the network 110 for storage at the user data store 314.

Next, at block 504, the calibrated images can be analyzed for any suitable application, including any of those set forth above. For example, the calibrated images can be analyzed to determine a skin condition of the area of interest. In some embodiments, the skin condition engine 208 of the mobile computing device 104 or the skin condition engine 306 of the server computing device 108 analyzes the calibrated images and determines, for example, acne, age spots, dry patches, etc., for each region of the area of interest. In doing so, the skin condition engine can access data from the skin condition data store 218, 318.

The example of the method 500 then proceeds to block 506, where an optional treatment protocol and/or product is for each region of the area of interest is recommended based on the determined skin condition (e. g., acne, dry skin, age spots, etc.). In some embodiments, the recommendation engine 212 of the mobile computing device 104 or the recommendation engine 312 of the server computing device 108 recommends a treatment protocol and/or product for each region of the area of interest based on the determined skin condition(s). In doing so, data can be accessed from the product data store 216, 316. Different products and/or treatment protocols can be recommended for regions with difference skin conditions. Any recommendation generated by the recommendation engine can be presented to the user in any fashion via the user interface engine on display 202. In some embodiments, the efficacy of the recommendation can be tracked, which can be used to train the recommendation engine and/or data stored in the product data store for improved recommendations in subsequent uses.

Of course, any processing accomplished at the mobile computing device 104 can be additionally or alternatively carried out at the server computing device 108.

The method 500 then proceeds to an end block and terminates.

Other embodiments are contemplated. For example, the calibration device and/or mobile computing device could also include positional sensors and inertial measurement sensors for generating additional data to be used to calibrate the images.

The present application may reference quantities and numbers. Unless specifically stated, such quantities and numbers are not to be considered restrictive, but exemplary of the possible quantities or numbers associated with the present application. Further in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Throughout this specification, terms of art may be used. These terms are to take on their ordinary meaning in the art from which they come, unless specifically defined herein or the context of their use would clearly suggest otherwise.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

1. A computer implemented method for accurate skin diagnosis, comprising:
calibrating, by a computing device, one or more images of an area of interest associated with a subject; and
determining a skin condition based on the one or more calibrated images.

2. The computer implemented method of claim 1, wherein the one or more images includes a plurality of images taken sequentially over a period of time, and wherein said determining a skin condition is based on the plurality of images.

3. The computer implemented method of claim 2, further comprising
generating a treatment protocol and/or product recommendation for an area of interest of the subject based on the determined skin condition.

4. The computer implemented method of claim 2, wherein said calibrating, by a computing device, one or more images of an area of interest associated with a subject includes:
obtaining calibration data from a calibration device;
calibrating a camera based on the calibration data; and
capturing the one or more images of a user with the calibrated camera.

5. The computer implemented method of claim 4, further comprising:
generating, via the calibration device, light meter data or color temperature data of the subject;
receiving the calibration data from the calibration device; and
adjusting one or more camera settings for calibrating the camera prior to image capture.

6. The computer implemented method of claim 2, wherein said calibrating, by a computing device, one or more images of an area of interest associated with a subject includes:
capturing the one or more images via a camera associated with the computing device;
obtaining calibration data from a calibration device associated with the one or more images captured by the camera; and
calibrating the one or more images captured by the camera based on the calibration data.

7. The computer implemented method of claim 6, further comprising:
generating, via the calibration device, light meter data or color temperature data of the subject;
receiving the light meter data and/or color meter data from the calibration device; and
using said light meter data and/or color meter data obtained from the calibration device to calibrate the captured images.

8. The computer implemented method of claim 6, wherein the calibration device includes one selected from the group consisting of a color card, a color chip and a calibration reference, the method further comprising capturing at least one image of the subject in the presence of the calibration device.

9. The computer implemented method of claim 8, wherein the calibration device is a cosmetics apparatus.

10. A method, comprising:
obtaining calibration data from a calibration device;
generating, by a computing device, calibrated images by one of:
calibrating a camera of a mobile computing device based on the calibration data and capturing one or more images of a user with the calibrated camera; or
calibrating one or more images captured with the camera based on the calibration data.

11. The method of claim 10, further comprising
determining a skin condition based on the one or more calibrated images.

12. The method of claim 11, further comprising
recommending one or more of: a skin treatment protocol; and a product configured to treat the skin condition.

13. A computer system, comprising:
a user interface engine including circuitry configured to cause an image capture device to capture images of the user;
a calibration engine including circuitry configured to calibrate the image capture device prior to image capture for generating calibrated images or to calibrate the images captured by the image capture device for generating calibrated images, said calibration engine obtaining calibration data from a calibration device; and
a skin condition engine configured to determine a skin condition of the user based on the generated calibrated images image.

14. The computer system of claim 13, further comprising
a recommendation engine including circuitry configured to recommend a treatment protocol or a product based at least on the determined skin condition.

15. The computer system of claim 14, wherein the calibration device includes one or more sensors configured to generate data indicative of calibration data, and wherein the calibration engine is configured to receive the calibration data and adjust one or more suitable camera settings for calibrating the camera prior to image capture.

16. The computer system of claim 13, wherein the calibration device includes an attribute suitable for use by the calibration engine to generate the calibrated images.

17. The computer system of claim 16, wherein the attribute is a color or an indicia indicative of a color, the calibration engine configured to obtain calibration data based on the indicia.

18. The computer system of claim 17, wherein the calibration engine includes circuitry configured to obtain the calibration data from the image captured by the image capture device, the image captured including an image of the calibration device.

19. The computer implemented method of claim 18, wherein the calibration device is a cosmetics apparatus or packaging associated therewith.

20. The computer implemented method of claim 18, wherein the calibration engine is configured to:
automatically detect a color reference associated with the captured image; and
use the color reference in order to correct the colors of the captured image to generate calibrated images.
